SEQUENCE LISTING (5 sequences)

SEQ ID NO: 1 - protein, 13 aa, synthetic construct
  source 1..13: note = SpyTag: amino acid sequence
AHIVMVDAYKPTK

SEQ ID NO: 2 - protein, 134 aa, synthetic construct
  source 1..134: note = SpyCatcher amino acid sequence
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATK

SEQ ID NO: 3 - protein, 5 aa, synthetic construct
  source 1..5: note = Sortase-coupling
LPXTG

SEQ ID NO: 4 - protein, 4 aa, synthetic construct
  source 1..4: note = Sortase-coupling
GGGG

SEQ ID NO: 5 - protein, 5 aa, synthetic construct
  source 1..5: note = Sortase-coupling
GGGGG

(19) United States
(12) Patent Application Publication
(10) Pub. No.: US 2025/0115957 A1
(43) Pub. Date: Apr. 10, 2025

(54) ON-SEQUENCER IMAGING FLOWCELL REUSE

(71) Applicant: Illumina, Inc., San Diego, CA (US)

(72) Inventors: Claudia Battistella, San Diego, CA (US); Gabriele Canzi, San Diego, CA (US); Katharina Mueller-Ott, Saffron Walden (GB); Wayne George, London (GB); Jonathan Boutell, Bishops Stortford (GB)

(73) Assignee: Illumina, Inc., San Diego, CA (US)

(21) Appl. No.: 18/897,718

(22) Filed: Sep. 26, 2024

Related U.S. Application Data
(60) Provisional application No. 63/586,350, filed on Sep. 28, 2023.

(51) Int. Cl. C12Q 1/6874

(52) U.S. Cl. CPC C12Q 1/6874

(57) ABSTRACT

Some examples herein provide a sequencing flowcell that includes an imaging sensor; a hydrogel disposed on the imaging sensor and comprising a moiety; and a first complex non-covalently coupled to the moiety, the first complex comprising a first oligonucleotide. A method of using the sequencing flowcell may include decoupling the first complex from the moiety; and coupling a second complex to the moiety, the second complex comprising a second oligonucleotide. Methods of forming the flowcell are also provided.

### CROSS-REFERENCE TO RELATED APPLICATIONS

This application claims the benefit of U.S. Provisional 63/586,350, filed Sep. 28, 2023 and entitled “On-Sequencer Imaging Flowcell Reuse,” the entire contents of which are incorporated by reference herein.

### SEQUENCE LISTING

The instant application contains a Sequence Listing which has been submitted electronically in XML format and is hereby incorporated by reference in its entirety. The accompanying sequence listing XML file, named “IP-2656-US.xml”, was created on Dec. 19, 2024 and is 6.2 KB in size.

### FIELD

This application relates to sequencing flowcells.

### BACKGROUND

A significant amount of academic and corporate time and energy has been invested into using flowcells to sequence polynucleotides. However, such previously known devices, systems, and methods may not necessarily be sufficiently sustainable or cost-effective. For example, the single-use nature of flowcells may increase the cost of sequencing polynucleotides and create waste.

### SUMMARY

Examples herein relate to on-sequencer imaging flowcell reuse.

Some examples provide a method of forming a sequencing flowcell. The method may include, in a solution, coupling a hydrogel precursor to a molecule by covalently bonding a first moiety of the hydrogel precursor to a second moiety of the molecule. The method may include disposing the solution, including the hydrogel precursor coupled to the molecule, on a substrate to form a hydrogel disposed on the substrate. The method may include, with the hydrogel disposed on the substrate, non-covalently bonding a third moiety of the molecule to a complex including an oligonucleotide.

In some examples, the substrate includes an image sensor over which the hydrogel is disposed. In some examples, the image sensor includes a complementary metal oxide semiconductor (CMOS) sensor.

In some examples, the substrate includes tantalum oxide (TaOₓ).

In some examples, the complex includes a protein with a first active site to which the oligonucleotide is coupled, and a second active site that non-covalently bonds to the third moiety.

Some examples herein provide a sequencing flowcell that includes an imaging sensor; a hydrogel disposed on the imaging sensor and including a moiety; and a first complex non-covalently coupled to the moiety, the first complex including a first oligonucleotide.

Some examples herein provide a method of using such a sequencing flowcell. The method may include decoupling the first complex from the moiety; and coupling a second complex to the moiety, the second complex including a second oligonucleotide.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1A illustrates a flow of example operations for using and reusing an imaging flowcell, and example structures formed using such operations.

FIG. 1B illustrates a nonlimiting example of elements that may be used in the example of FIG. 1A.

FIG. 2 illustrates a flow chart showing the flow of operations in an example automated method conducted in an imaging flowcell.

FIG. 3A is a plot of coating thickness of example polymers before and after rinse on model unpatterned TaOx slides.

FIG. 3B is a plot of dry vs. wet thickness of example polymers as measured by ellipsometry measurements.

FIGS. 4A-4C illustrate fluorescence images and intensities obtained following binding steps and stripping steps of example polymers.

FIG. 5 illustrates fluorescence images and intensities obtained after grafting oligonucleotides to example polymers.

FIG. 6 illustrates fluorescence images and intensities obtained after disposing an example polymer in nanowells of a CMOS substrate.

### DETAILED DESCRIPTION

Automated methods conducted in an imaging flowcell, and kits for reusing a flowcell, such as an imaging flowcell, are provided herein.

In order to help sequencing become more sustainable, the present subject matter is directed to reusing flowcells by applying different regeneration methods. Flowcell reuse is particularly useful in the case of expensive substrates, such as imaging flowcells. For example, flowcells that include complementary metal oxide semiconductor (CMOS) imaging sensors are particularly expensive to manufacture. As such, reusing imaging flowcells (such as CMOS-based imaging flowcells) can provide significant cost savings, and also reduce waste of valuable materials. To allow the end user to reuse imaging flowcells (such as CMOS-based flowcells) without requiring them to learn additional skills, some examples herein provide imaging flowcell reuse which is fully integrated in a sequencing run, with the imaging flowcell staying on-board the instrument and being regenerated either before or after each run. Described herein are methods and kits that focus on the implementation of regeneration steps on-board a sequencing instrument without additional touch points. While the chemistries and procedures described herein are compatible with imaging flowcells (such as CMOS-based imaging flowcells), it will be appreciated that they may be used with any other suitable type of flowcell, including flowcells that do not include an image sensor or devices for library preparation that would be benefit from reuse.

First, some terms used herein will be briefly explained. Then, some example methods for reuse conducted in an imaging flowcell, and example kits for reusing the imaging flowcell, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Substrates that may be used to generate an image may be referred to as an “imaging substrate.” In examples in which a flowcell includes an imaging substrate, the flowcell may be referred to as an “imaging flowcell.” Example substrate materials may include glass, silica, plastic, quartz, metal, metal oxide, organo-silicate (e.g., polyhedral organic silsesquioxanes (POSS)), polyacrylates, tantalum oxide (e.g., TaOₓ, such as Ta₂O₅), complementary metal oxide semiconductor (CMOS), or combinations thereof. CMOS substrates may include tantalum oxide. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flowcell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM). The process creates hydrogel regions used for sequencing that may be stable over sequencing runs with a large number of cycles. The covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells. Primers may be attached to hydrogel material. A solution including a plurality of target polynucleotides (e.g., a fragmented human genome or portion thereof) may then be contacted with the polished substrate such that individual target polynucleotides will seed individual wells via interactions with primers attached to the hydrogel material; however, the target polynucleotides will not occupy the interstitial regions due to absence or inactivity of the hydrogel material. Amplification of the target polynucleotides may be confined to the wells because absence or inactivity of hydrogel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate. A nonlimiting example of a hydrogel is a polyacrylamide polymer (such as PAZAM or SFA (e.g., azidoSFA).

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

In some examples, a substrate described herein forms at least part of a flowcell or is located in or coupled to a flowcell. Flowcells may include a flow chamber that is divided into a plurality of lanes or a plurality of sectors. Example flowcells and substrates for manufacture of flowcells that can be used in methods and compositions set forth herein include, but are not limited to, those commercially available from Illumina, Inc. (San Diego, CA).

As used herein, terms such as “covalently coupled” or “covalently bonded” refer to the forming of a chemical bond that is characterized by the sharing of pairs of electrons between atoms. For example, a covalently coupled molecule refers to a molecule that forms a chemical bond, as opposed to a non-covalent bond such as electrostatic interaction.

As used herein, the term “complex” is intended to refer to molecules that are coupled together to form a larger structure. In some examples, a complex may include a central molecule, such as a protein, to which a polynucleotide (such as a capture primer) is coupled. The polynucleotide may be coupled to the central molecule via reaction of a first moiety coupled to the polynucleotide, with a second moiety coupled to the central molecule. Such a reaction may form a covalent bond, or may form a noncovalent bond. As described in greater detail below, the central molecule of a complex may be reversibly coupled to a moiety at a surface.

As used herein, the term “linker” is intended to mean a portion of a molecule via which one element is attached to another element. For example, a linker may attach a first reactive moiety to a second reactive moiety. Linkers may be covalent.

### Automated Methods Conducted in an Imaging Flowcell, and Kits for Reusing an Imaging Flowcell

As will be discussed below, some of the disclosed methods involve strategies for reusing a flowcell, such as an imaging flowcell. One strategy leverages reversible linkages, such as those between biotin and streptavidin. For example, a surface including a first type of moiety (such as biotin) is generated by grafting a molecule containing that moiety onto the surface (e.g., onto the azides of a polymer coating at that surface). Next, a second type of moiety (e.g., a streptavidin-dualbiotin structure) is used to bind surface primers to the first type of moiety (e.g., biotin) on the surface and enable clustering and sequencing. For regeneration of the surface, the reversible linkage interactions (e.g., biotin-streptavidin interactions) are reversed using a reagent (e.g., hot formamide). A nuclease digest ensures that no DNA from one run will be carried over into the following run.

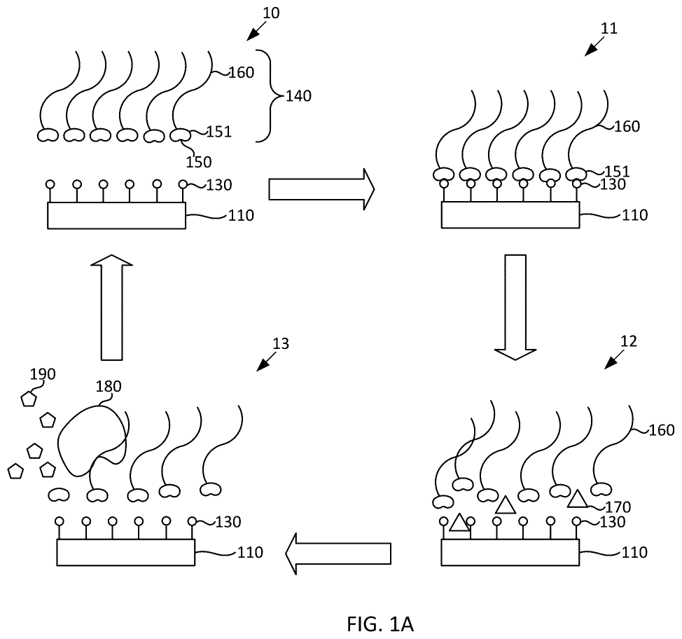

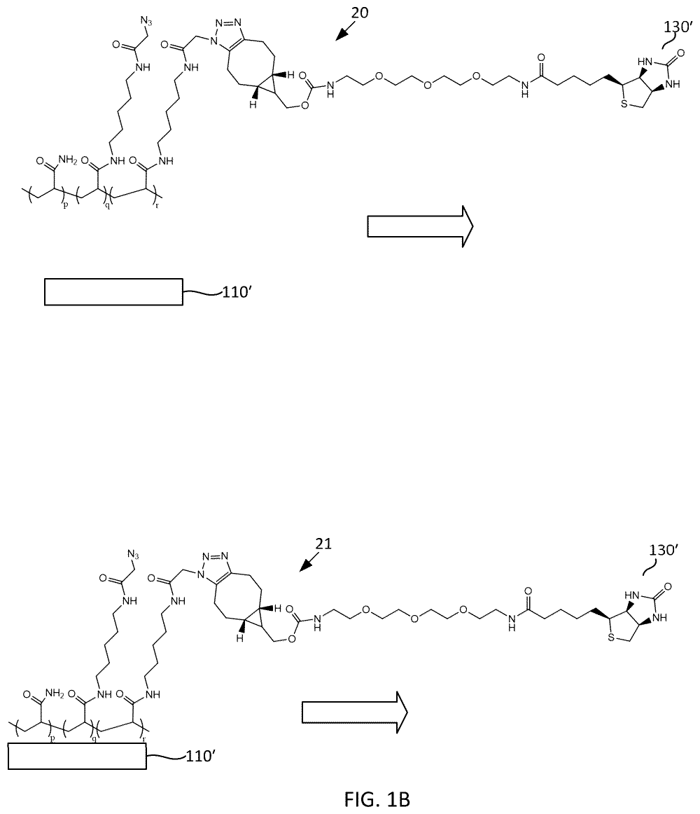

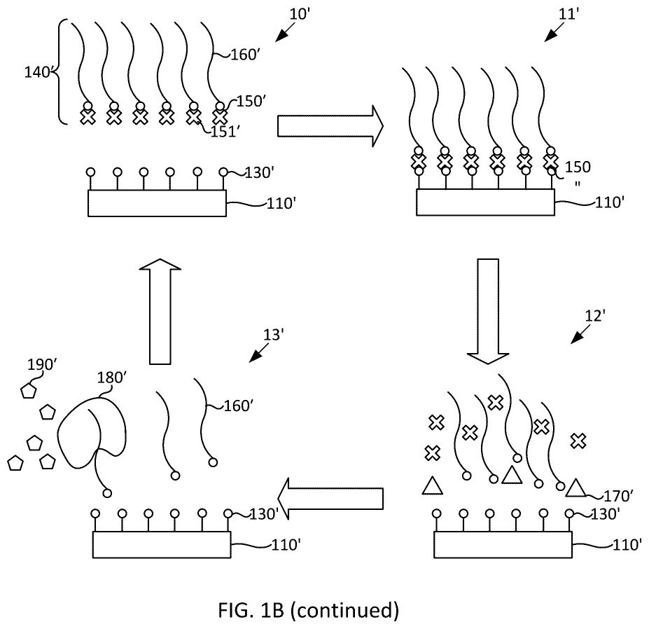

Illustratively, FIG. 1A illustrates a flow of example operations for using and reusing an imaging flowcell, and example structures formed using such operations. As illustrated at operation 10 of FIG. 1A, surface 110, including moieties 130, may be contacted with a fluid that includes complexes 140. Surface 110 may be in a flowcell, such as an imaging flowcell. Surface 110 may include a substrate. For example, surface 110 may include glass, silica, plastic, quartz, metal, metal oxide, organo-silicate (e.g., POSS), polyacrylates, tantalum oxide, CMOS, or combinations thereof. In one nonlimiting example, surface 110 may be or include tantalum oxide at the upper surface of a CMOS imaging flowcell. Nonlimiting examples of compositions and methods for preparing surface 110 including moieties 130 are described further below with reference to FIG. 1B.

Moiety 130 may be used to reversibly couple complex 140 to the surface 110, so as to reversibly couple an oligonucleotide to the surface in a manner such as illustrated at operation 11 of FIG. 1B. For example, complex 140 may include moiety 150 which couples to moiety 130, and at least one oligonucleotide 160 coupled to moiety 150. In some examples, complex 140 includes central molecule 151 (such as a protein), that includes one or more active sites to which other elements may be coupled. For example, oligonucleotide 160 may be coupled to a first active site of central molecule 151, e.g., via covalent or non-covalent interaction between a moiety coupled to the oligonucleotide and the first active site (in this, note that active sites may be considered moieties). In some examples, moiety 150 may correspond to a second active site on the central molecule 151.

Oligonucleotide 160 may be single-stranded, and optionally may be bound to moiety 150 via a suitable element, such as a linker. Oligonucleotide 160 may be or include a capture primer such as may be used for seeding and/or amplifying a template polynucleotide.

At operation 11, complex 140 may be coupled to surface 110 via reaction between moiety 130 coupled to surface 110 and moiety 150 of complex 140. Moiety 130 and moiety 150 may include any suitable pair of reactive moieties that couple to one another in a way that is substantially irreversible during certain operations such as described with reference to FIG. 1A (e.g., during seeding, amplifying, and sequencing template polynucleotides), and subsequently may be decoupled from one another during one or more other operations such as described with reference to operations 12 and 13 of FIG. 1A (e.g., when regenerating the flowcell for reuse after sequencing). In some examples, moiety 150 may form a non-covalent bond with moiety 130, which may facilitate subsequent removal of complex 140 from the flowcell so that the flowcell may be reused in a manner such as described herein.

In some examples, moiety 130 may be selected so as to bond to an active site (moiety 150) of molecule 151. For example, moiety 130 and molecule 151 may include a biotin/streptavidin pair, a polyhistidine-tag (His-tag)/transition metal pair, a DIG/anti-DIG pair, a c-myc/anti-cmyc pair, a GST/glutathione pair, or a FLAG/anti-FLAG pair. Non-limiting examples of pairs of moiety 130 and central molecule 151 that may be used to couple molecule 120 to complex 140 are shown in Table 1 below. Additionally, non-limiting examples of reagents that may be used to decouple molecule 120 from complex 140 in operation 12 (described in further detail below) are shown in Table 1.

TABLE 1

| | | | Example reagent(s) for |
|---|---|---|---|
| | | | decoupling moiety 130 |
| | Example | Example central | from central molecule |
| Bonding pair | moiety 130 | molecule 151 | 151 |
| biotin-streptavidin | biotin, | Streptavidin, | formamide or ethylene |
| | desthiobiotin, dual- | neutravidin, avidin, | glycol |
| | biotin, Strep-tag | Strep-Tactin | |
| His-tag transition | transition metal | Histidine tag (His-tag) | imidazole |
| metal | (e.g., Mn²⁺, Fe²⁺, | | |
| | Co₂₊, Ni²⁺, or Cu²⁺) | | |
| His-tag transition | His-tag | transition metal (e.g., | imidazole |
| metal | | Mn²⁺, Fe²⁺, Co₂₊, Ni²⁺ | |
| | | or Cu²⁺) | |
| DIG/anti-DIG | digoxigenin (DIG) | anti-digoxigenin (anti- | Glycine approx. pH 3.0 |
| | | DIG) antibody | or approx. 3M sodium |
| | | | thiocyanate (NaSCN) |
| c-myc/anti-cmyc | c-myc (also referred | anti-cmyc antibody | Glycine approx. pH 3.0 |
| | to as MYC) | | or approx. 3M sodium |
| | | | thiocyanate (NaSCN), or |
| | | | competition with c-myc |
| | | | peptide |
| c-myc/anti-cmyc | anti-cmyc antibody | c-myc | Competition with c-myc |
| | | | peptide |
| GST/glutathione | glutathione | glutathione s-transferase | Competition with excess |
| | | (GST) | glutathione |
| GST/glutathione | glutathione s- | glutathione | Competition with excess |
| | transferase (GST) | | glutathione |
| FLAG/anti-FLAG | FLAG tag | Anti-FLAG antibody | Glycine approx. pH 3.5 |
| | | | or competition with |
| | | | excess FLAG peptide |
| FLAG/anti-FLAG | Anti-FLAG | FLAG tag | Competition with excess |
| | antibody | | FLAG peptide |

In one nonlimiting example, molecule 151 is or includes a protein having multiple active sites (moieties 150). Oligonucleotide 160 may be coupled to a moiety that is coupled to a first one of the active sites (e.g., via its own moiety 130 such as exemplified in Table 1), and moiety 130 of molecule 120 may be coupled to a second one of the active sites. Illustratively, molecule 151 may be or include streptavidin or related protein (e.g., neutravidin, avidin, or Strep-Tactin); oligonucleotide 160 may be coupled to biotin or related moiety (e.g., desthiobiotin, dual-biotin, or Strep-tag) that is coupled to a first active site of the molecule 151; and moiety 130 may be or include another biotin or related moiety (e.g., desthiobiotin, dual-biotin, or Strep-tag) that is coupled to a second active site of the molecule 151. Optionally, complex 140 may include multiple oligonucleotides 160, e.g., coupled to different active sites of molecule 151. The oligonucleotides may have the same sequences as one another (e.g., may all be P5, or may all be P7). Alternatively, the oligonucleotides may have different sequences than one another (e.g., may be or include a mixture of P5 and P7). In nonlimiting examples where molecule 151 includes streptavidin or related protein, the protein may be incubated ahead of time with multiple P5 and/or P7 oligonucleotides 160 which are coupled to respective biotins or related moieties to form a complex which may referred to herein as “streptavidin-dualbiotin-P5/P7”. In some examples, the resulting complex 140 may include one, two, or three oligonucleotides 160, leaving at least one available active site available to bind with biotin or related moiety at surface 110 in a manner such as illustrated in FIG. 1A. As another example, molecule 151 may be or include His-tag which is coupled directly or indirectly to oligonucleotide 160, and moiety 130 may be or include a transition metal that is coupled to an active site of the His-tag. As another example, molecule 151 may be or include a transition metal which is coupled directly or indirectly to oligonucleotide 160, and moiety 130 may be or include a His-tag with an active site that is coupled to the transition metal. It will be appreciated that any suitable combination of coupling options may be used, at least some of which are expected to be orthogonal.

After operation 11 is performed, oligonucleotides 160 may be used for seeding, clustering, and sequencing processes (not specifically illustrated), e.g., may be used as primers to generate clusters of amplicons that may be sequenced using sequencing-by-synthesis. In examples in which the flowcell is an imaging flowcell, the flowcell may include an image sensor that detects fluorescence during sequencing-by-synthesis from which the identities of different bases may be determined. Following such sequencing, the flowcell may be regenerated and then reused. For example, at operation 12 illustrated in FIG. 1A, reagent 170 may be introduced to decouple complex 140 from moiety 130. For example, reagent 170 may interfere with the bond between moiety 150 and moiety 130 (illustratively, by denaturing central molecule 151, or by competing with the bond between moiety 150 and moiety 130) thereby decoupling complex 140 from moiety 130.

At operation 13 illustrated in FIG. 1A, a nuclease digest is also performed such that nuclease 180 digests polynucleotides in the flowcell (e.g., oligonucleotides 160, and any polynucleotides coupled thereto), into nucleotides 190. Nuclease 180 may include any appropriate nuclease. For example, nuclease 180 may include a polymerase, illustratively a DNA polymerase that has 3′ to 5′ exonuclease activity. Additionally, or alternatively, nuclease 180 may include an exonuclease (such as Exonuclease I, also referred to as ExoI). Additionally, or alternatively, nuclease 180 may include a non-specific dsDNA nuclease (such as DNaseI). Additionally, or alternatively, nuclease 180 may include Micrococcal Nuclease (MNase). MNase digests 5′-phosphodiester bonds of DNA and RNA, yielding 3′-phosphate mononucleotides and oligonucleotides. For further details regarding MNase, see the following references, the entire contents of which are incorporated by reference herein: Cuatrecasas et al., “Catalytic properties and specificity of the extracellular nuclease of Staphylococcus aureus,” J. Biol. Chem. 242(7): 1541-1547 (1967); Craig et al., “Plasmid cDNA-directed protein synthesis in a coupled eukaryotic in vitro transcription-translation system,” Nucleic Acids Res. 20(19): 4987-4985 (1992); and O'Neill et al., “Immunoprecipitation of native chromatin: NChIP,” Methods 31(1): 86-82 (2003). In some examples, nuclease 180 may include a combination of different nucleases, such as a combination of DNaseI and ExoI. In other examples, nuclease 180 may consist essentially of MNase. Illustratively, the inventors have observed that MNase is particularly potent, giving significantly less contamination from run to run when compared to a combination of DNaseI and ExoI (so the single enzyme MNase may in some examples perform better than the 2 enzymes together).

At any suitable time after using reagent 170 (operation 12) and nuclease 180 (operation 13), the surface 110 may be washed, a new set of molecules 140 can be introduced (operation 10), and the cycle repeated. The new set of complexes 140 may be of the same type or of a different type than the original set of complexes 140. Illustratively, the new set of complexes may include oligonucleotides having the same sequence as oligonucleotides 160, or having one or more different sequences than oligonucleotides 160.

FIG. 1B illustrates a nonlimiting example of elements that may be used in the example of FIG. 1A, in which the use of the prime symbol (′) for a given element denotes that this element in FIG. 1B is a nonlimiting example of an implementation of the corresponding element of FIG. 1A. Referring now to operation 20 of FIG. 1B, surface 110′ may be or include the surface of an imaging sensor, e.g., a CMOS imaging sensor, including tantalum oxide. As shown at operation 20 of FIG. 1B, surface 110′ may be contacted with a fluid that contains a hydrogel, illustratively a polyacrylamide polymer (such as PAZAM or SFA) to which moiety 130′ has been coupled prior to such contact. Surface 110′ may be suitably functionalized before contacting it with the fluid, e.g., may be silanized using norbornene so as to bind the azide of an azide-containing polymer. In the example illustrated in FIG. 1B, moiety 130′ is biotin, but it will be appreciated that any other suitable moieties 130, such as described in Table 1, may be used in place of the biotin. The hydrogel (e.g., polyacrylamide polymer) may be formed in any suitable manner. Illustratively, as shown in the scheme below, the hydrogel may be formed to include a first moiety (e.g., —N₃ in this nonlimiting example), and the biotin is coupled to a second moiety (e.g., BCN in this nonlimiting example) that reacts with the first moiety to couple the biotin to the hydrogel. Note that other conditions (e.g., other temperatures, such as any temperature between room temperature and 65° C. or more) suitably may be used.

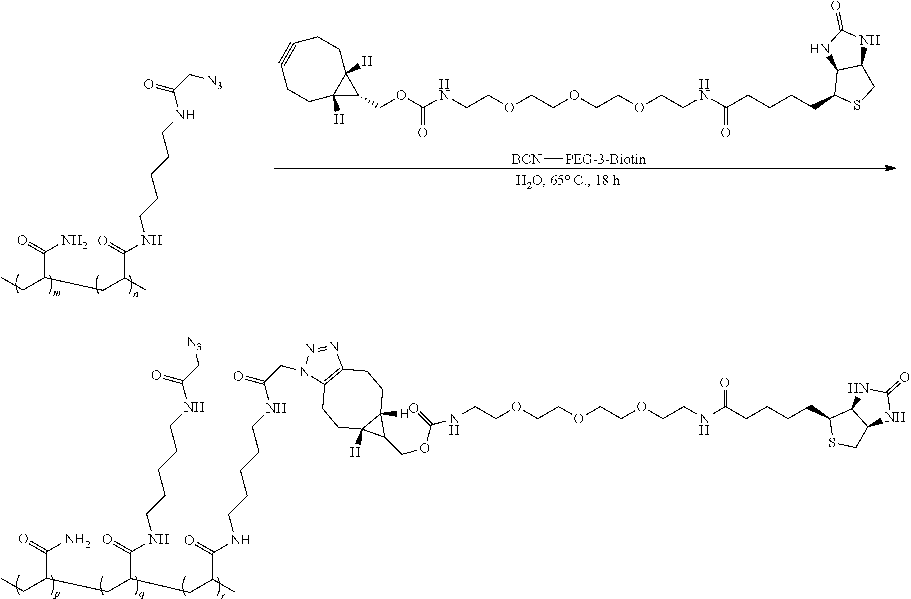

It will be appreciated that the first moiety coupled to the hydrogel, and the second moiety coupled to the biotin (or other moiety 130 from Table 1) may include any suitable pair of reactive moieties that form a linkage which is substantially irreversible during operations such as described with reference to FIG. 1B. For example, the first moiety and the second moiety may include an amine-NHS pair, an amine-imidoester pair, an amine-pentofluorophenyl ester pair, an amine-hydroxymethyl phosphine pair, an amine-carboxylic acid pair, a thiol-maleimide pair, a thiol-haloacetyl pair, a thiol-pyridyl disulfide pair, a thiol-thiosulfonate pair, a thiol-vinyl sulfone pair, an aldehyde-hydrazide pair, an aldehyde-alkoxyamine pair, a hydroxy-isocyanate pair, an azide-alkyne pair, an azide-phosphine pair, an azide-cyclooctyne pair, an azide-norbornene pair, a transcycloctene-tetrazine pair, a norbornene-tetrazine pair, an oxime, a SpyTag-SpyCatcher pair, a SNAP-tag-O⁶-benzylguanine pair, a CLIP-tag-O²-benzylcytosine pair, or a sortase coupling. Non-limiting examples of moiety pairs that may be used to couple the first moiety (coupled to the hydrogel, e.g., polyacrylamide polymer) to the second moiety (coupled to moiety 130, such as biotin 130′) are shown in Table 2 below.

TABLE 2

| | Example first or second | |
|---|---|---|
| Bonding pair | moiety | Example second or first moiety |
| amine-NHS | amine group, —NH₂ | N-Hydroxysuccinimide ester |
| | | 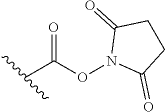 [SMILES: *C(=O)ON1C(=O)CCC1=O] |
| amine-imidoester | amine group, —NH₂ | imidoester |
| | | 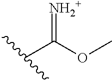 [SMILES: *C(=[NH2+])OC] |
| amine- | amine group, —NH₂ | pentofluorophenyl ester, |
| pentofluorophenyl | | |
| ester | | 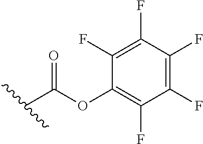 [SMILES: *C(=O)Oc1c(F)c(F)c(F)c(F)c1F] |
| amine- | amine group, —NH₂ | hydroxymethyl phosphine |
| hydroxymethyl | | |
| phosphine | | 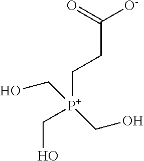 [SMILES: O=C([O-])CC[P+](CO)(CO)CO] |
| amine-carboxylic | amine group, —NH₂ | carboxylic acid group, —C(═O)OH (e.g., |
| acid | | following activation of the carboxylic |
| | | acid by a carbodiimide such as EDC (1- |
| | | ethyl-3-(-3-dimethylaminopropyl) |
| | | carbodiimide hydrochloride) or DCC |
| | | (N′,N′-dicyclohexyl carbodiimide) to |
| | | allow for formation of an amide bond |
| | | of the activated carboxylic acid with an |
| | | amine group) |
| thiol-maleimide | thiol, —SH | maleimide |
| | | 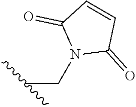 [SMILES: *CN1C(=O)C=CC1=O] |
| thiol-haloacetyl | thiol, —SH | haloacetyl (e.g., iodoacetyl or other |
| | | haloacetyl) |
| | | 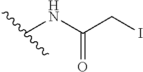 [SMILES: *NC(=O)CI] |
| thiol-pyridyl | thiol, —SH | pyridyl disulfide |
| disulfide | | |
| | | 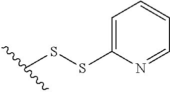 [SMILES: *SSc1ccccn1] |
| thiol-thiosulfonate | thiol, —SH | thiosulfonate |
| | | 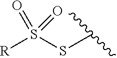 |
| thiol-vinyl sulfone | thiol, —SH | vinyl sulfone |
| | | 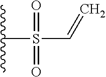 [SMILES: *S(=O)(=O)C=C] |
| aldehyde-hydrazide | aldehyde, —C(═O)H | hydrazide |
| | | 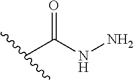 [SMILES: *C(=O)NN] |
| aldehyde- | aldehyde, —C(═O)H | alkoxyamine |
| alkoxyamine | | |
| | | 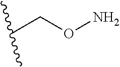 [SMILES: *CON] |
| hydroxy-isocyanate | hydroxyl, —OH | isocyanate |
| | | 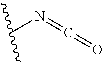 [SMILES: *N=C=O] |
| azide-alkyne | azide, —N₃ | alkyne |
| | | 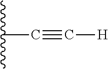 [SMILES: *C#C] |
| azide-phosphine | azide, —N₃ | phosphine, e.g.: |
| | | 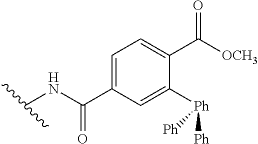 |
| azide-cyclooctyne | azide, —N₃ | cyclooctyne, e.g. dibenzocyclooctyne |
| | | (DBCO) |
| | | 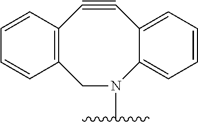 [SMILES: *N1Cc2ccccc2C#Cc2ccccc21] |
| | | or BCN (bicyclo[6.1.0]nonyne) |
| | | 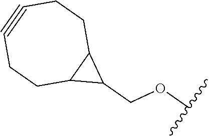 [SMILES: *OCC1C2CCC#CCCC21] |
| azide-norbornene | azine, —N₃ | norbornene |
| | | 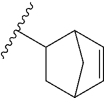 [SMILES: *C1CC2C=CC1C2] |
| transcyclooctene- | Transcyclooctene (axial, | tetrazine, e.g., benzyl-methyltetrazine |
| tetrazine | equatorial) | |
| | 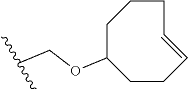 [SMILES: *COC1CC/C=C/CCC1] | 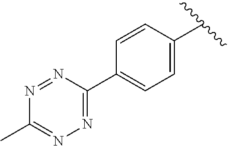 [SMILES: *c1ccc(-c2nnc(C)nn2)cc1] |
| norbornene- | norbornene | tetrazine, e.g. benzyl-tetrazine |
| tetrazine | | |
| | 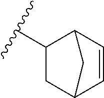 [SMILES: *C1CC2C=CC1C2] | 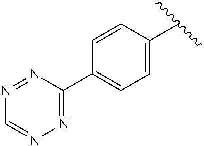 [SMILES: *c1ccc(-c2nncnn2)cc1] |
| oxime | aldehyde or ketone (e.g., amine | alkoxyamine |
| | group or N-terminus of | |
| | polypeptide converted to an | |
| | aldehyde or ketone by pyroxidal | |
| | phosphate) | |
| SpyTag- | SpyTag: amino acid sequence | SpyCatcher amino acid sequence: |
| SpyCatcher | AHIVMVDAYKPTK (SEQ ID | MKGSSHHHHHHVDIPTTENLYFQ |
| | NO: 1) | GAMVDTLSGLSSEQGQSGDMTIEE |
| | | DSATHIKFSKRDEDGKELAGATME |
| | | LRDSSGKTISTWISDGQVKDFYLY |
| | | PGKYTFVETAAPDGYEVATAITFT |
| | | VNEQGQVTVNGKATK (SEQ ID |
| | | NO: 2) |
| SNAP-tag-O⁶- | SNAP-tag (O-6-methylguanine- | O⁶-Benzylguanine |
| Benzylguanine | DNA methyltransferase) | |
| | | 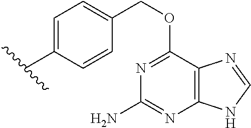 [SMILES: *c1ccc(COc2nc(N)nc3[nH]cnc23)cc1] |
| CLIP-tag-O²- | CLIP-tag (modified O-6- | O²-benzylcytosine |
| benzylcytosine | methylguanine-DNA | |
| | methyltransferase) | 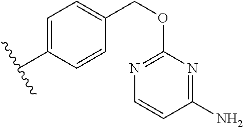 [SMILES: *c1ccc(COc2nccc(N)n2)cc1] |
| Sortase-coupling | -Leu-Pro-X-Thr-Gly (SEQ ID | -Gly₍₃₋₅₎ (SEQ ID NO: 4) (SEQ ID |
| | NO: 3) | NO: 5) |

In the nonlimiting example in the chemical scheme above, and as shown at operation 20 of FIG. 1B, moiety 130′ optionally may be coupled to the second moiety of that molecule via a suitable element, such as a linker. Nonlimiting examples of linkers include an alkyl chain or a polymer. Nonlimiting examples of polymers for use in the linker include polyether, polyamide, polyester, polyaryl, poly(ethylene glycol) (PEG), or the like. Illustratively, the linker may include PEG having between 1 and 10 ethylene glycol units, e.g., PEG1 to PEG10, illustratively PEG2 to PEG6, such as PEG3 (the nonlimiting example shown in the chemical scheme above). In the nonlimiting example in which PEG3 is the linker, the molecules containing biotin may be referred to as alkyne-PEG3-biotin molecules. However it will be appreciated that linkers that include longer or shorter PEG chains, or even no PEG (example with no PEG shown below) suitably may be used. In some examples, one or more biotin moieties per linker (for instance dual biotin as shown below) may be used. In one nonlimiting example, a dualbiotin structure such as illustrated below may be used that includes first and second biotin moieties separated from one another by a PEG-free linker. This structure also or alternatively may be included in the monomer/polymer structure.

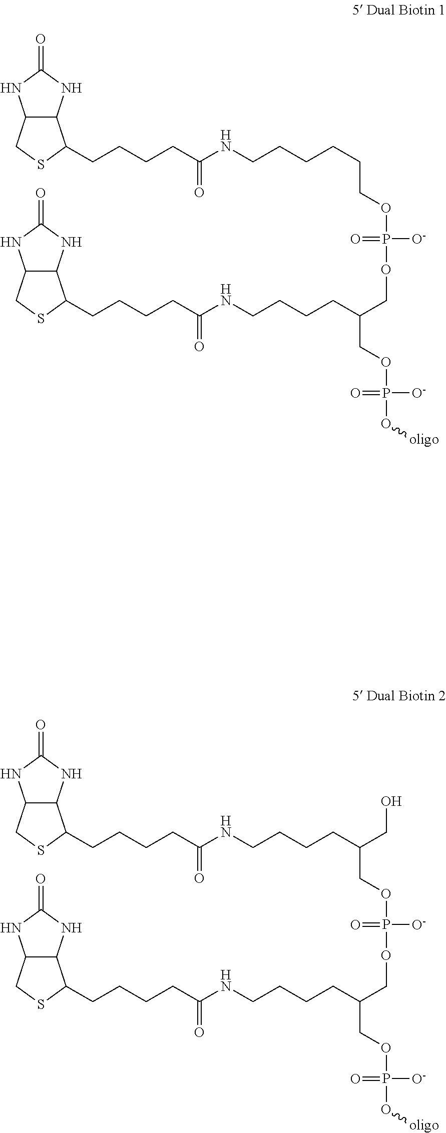

As shown at operation 21 of FIG. 1B, the hydrogel with biotin 130′ coupled thereto is disposed on surface 110′. Once attached to surface 110′ via the hydrogel, biotin 130′ is available to be reacted. In the nonlimiting example shown at operation 11′ of FIG. 1B, biotin 130′ then may be contacted with a fluid including pre-incubated streptavidin-dualbiotin-P5/P7 complexes 140′. Streptavidin-dualbiotin-P5/P7 complexes 140′ respectively include a streptavidin central molecule 151′ and at least one oligonucleotide 160′, e.g., one or more P5 and/or P7 oligonucleotides which are functionalized to dualbiotin to which a respective active site (moiety 150′) of streptavidin binds. As shown at operation 12′ of FIG. 1B, the biotin is then reacted with an active site 150″ of a pre-incubated streptavidin-dualbiotin-P5/P7 complex 140′, binding complexes 140′ to surface 110′. At this point, oligonucleotides 160′ can be used for appropriate clustering and sequencing processes (not specifically illustrated). To initiate regeneration and reuse of the flowcell, at operation 13′ a reagent such as hot formamide or ethylene glycol 170′ is introduced to decouple streptavidin central molecule 151′ from biotin 130′, for example by denaturing the streptavidin. Other example reagents for use in decoupling other pairs of elements are provided in Table 1. At operation 14′ of FIG. 1B, a nuclease digest is also performed such that nuclease 180′ digests polynucleotides in the flowcell, e.g., oligonucleotides 160′ and any polynucleotides coupled thereto, into nucleotides 190′. At this point, or at a later time, another set of complexes 140′ can be introduced (operation 10′) and the cycle repeated.

Additionally, kits are provided herein that include one or more elements such as described with reference to FIG. 1A or FIG. 1B. For example, a kit may include a plurality of complexes 140. Each complex 140 includes second moiety 150, which can couple to first moiety 130. Each complex 140 also includes oligonucleotide 160 which is coupled to second moiety 150. The kit may include a reagent 170 to decouple first moiety 130 and second moiety 150 from one another. The kit may include at least one nuclease 180 to digest polynucleotides. The flowcell may come with a surface that already includes first moiety 130 which is ready to be coupled to complexes 140 in the kit.

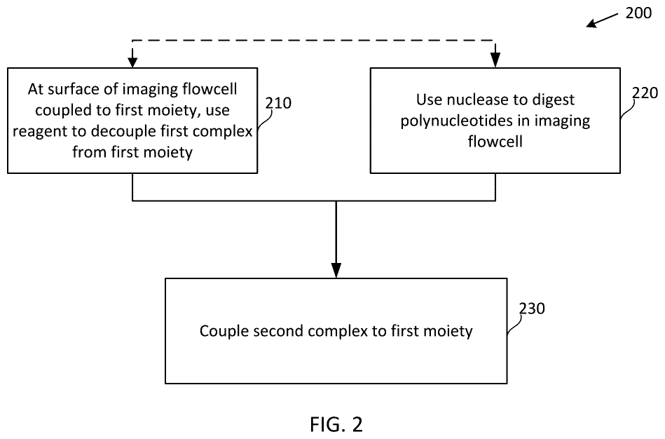

FIG. 2 illustrates a flow chart showing the flow of operations in an example automated method 200 conducted in an imaging flowcell. Method 200 may include, at the surface of the imaging flowcell coupled to a first moiety, using a reagent to decouple a first complex from the first moiety (operation 210). Nonlimiting examples of the reagent used at operation 210 are provided in Table 1 for different central molecules 151 and different moieties 130 that may be coupled to the flowcell. The first moiety referred to in operation 210 may correspond to moiety 130 described with reference to FIG. 1A, illustratively moiety 130′ described with reference to FIG. 1B. The first complex may correspond to complex 140 described with reference to FIG. 1A, illustratively complex 140′ described with reference to FIG. 1B. The first complex may include a second moiety which couples to the first moiety. In some examples, the second moiety may couple to the first moiety via a non-covalent bond. The second moiety may correspond to moiety 150 as described with reference to FIG. 1A, illustratively moiety 150′ described with reference to FIG. 1B. The regent may work, for example, by denaturing at least part of complex 140 (illustratively, by denaturing central molecule 151, such as in the example described with reference to FIG. 1B), or by competing with moiety 130 to bond to moiety 150.

The first complex referred to in operation 210 may also include a polynucleotide coupled to the second moiety. The polynucleotide may include, or may correspond to, oligonucleotide 160 as described with reference to FIG. 1A, illustratively oligonucleotide 160′ described with reference to FIG. 1B. The polynucleotide may have been used for appropriate clustering and sequencing processes, and as such may include oligonucleotide 160 or 160′ which is extended to include an amplicon of a template polynucleotide. The imaging flowcell may be used to detect optical signals from labeled nucleotides during the sequencing processes. Method 200 may also include using a nuclease to digest polynucleotides in the imaging flowcell (operation 220). For example, the nuclease may digest the polynucleotide coupled to the second moiety or a different polynucleotide in the flowcell. It should be noted that operation 210 may be performed either before or after operation 220, or even at the same time as operation 220, as intended to be indicated by the dashed arrow. Wash steps may be performed before and/or after each of operations 210 and 220. For example, operation 210 may be performed, the surface washed to remove decoupled complexes, operation 220 then performed to digest any remaining polynucleotides, and the surface washed again to remove digested polynucleotides. Or, for example, operation 220 may be performed to digest polynucleotides, the surface washed to remove digested polynucleotides, operation 210 may be performed to decouple complexes (from which the polynucleotides may have been at least partially removed), and the surface washed again to remove the decoupled complexes.

Method 200 may also include, after using the reagent and after using the nuclease, coupling a second complex to the first moiety (operation 230). The second complex 140 or 140′ may include a third moiety 150 or 150′ which couples with the first moiety 130 or 130′ and an oligonucleotide 160 or 160′ coupled to the third moiety. In some examples, the third moiety 150 or 150′ may couple to the first moiety 130 or 130′ via a non-covalent bond. Nonlimiting examples of moieties 130, 130′, 150, and 150′ are provided elsewhere herein. In some examples, the second moiety (e.g., 150 or 150′ of the first complex) is coupled to the first moiety 130 or 130′ via a first non-covalent bond, and the third moiety (e.g., 150 or 150′ of the second complex) is coupled to the first moiety 130 or 130′ via a second non-covalent bond).

In some examples, the surface may be prepared by disposing thereon a hydrogel (e.g., polyacrylamide polymer) which is pre-coupled to the first moiety, e.g., in a manner such as described with reference to FIG. 1B. Nonlimiting examples of moieties are provided elsewhere herein, e.g., with reference to Table 1.

In some examples, method 200 may further include, before using the reagent and before using the nuclease, sequencing the polynucleotide. For example, in a manner such as described with reference to FIGS. 1A and 1B, oligonucleotide 160 or 160′ may be used for seeding and/or amplifying a template polynucleotide, illustratively using sequencing-by-synthesis, during which the imaging flowcell may detect optical signals from which the sequence of the template polynucleotide may be determined. Illustratively, method 200 may further include, after coupling the second complex to the first moiety, using the oligonucleotide to amplify a template polynucleotide, and sequencing the amplified template polynucleotide. The flowcell may be regenerated and reused at any suitable time after the sequencing, and optionally stored at any suitable time. The regenerated flowcell may be stored in a suitably controlled environment to inhibit degradation of the moieties which are coupled to the surface at the time of storage.

For example, the flowcell may be washed and optionally stored after the second complex is coupled to the first moiety, and before the oligonucleotide is used to amplify the template polynucleotide. In this case, the flowcell may be ready for the next sequencing run as soon as the previous run finishes, and can be stored in this ready state. Alternatively, the flowcell may be washed and optionally stored after the polynucleotide is sequenced, before using the reagent, before using the nuclease, and before the second complex is coupled to the first moiety. Performing decoupling, digesting, and rebinding steps at the beginning of the sequencing run may delay the start of clustering, but may reduce the amount of time for which reagent 170 may be exposed to air which otherwise may cause degradation of the reagent. As yet another alternative, the flowcell may be washed and optionally stored after the polynucleotide is sequenced, after using the reagent, after using the nuclease, and before the second complex is coupled to the first moiety. In this example, the decoupling and digesting are performed at the end of the sequencing run, leaving the flowcell surface clean. The flowcell then can be regenerated by coupling a fresh set of complexes to the surface at the beginning of the sequencing run. Such re-binding is expected to cause only a short delay at the beginning of the sequencing run, or no delay at all in the case of a multi-flowcell system.

Note that all operations besides storing the flowcell (which entails removing the flowcell from the sequencer and moving it to a storage area) may be performed automatically by the sequencer. For example, the sequencer may be configured to receive a kit such as described above, e.g., that includes the reagent, nuclease, and complexes, and may be configured to automatically flow such elements into the flowcell at appropriate times to couple the complexes to the flowcell surface before sequencing, to use the reagent to decouple the complexes from the flowcell surface after sequencing, and to use the nuclease to digest any remaining polynucleotides within the flowcell before coupling additional complexes to the surface.

### Working Example

The following example is intended to be purely illustrative, and not limiting of the present invention.

A flowcell was prepared and used in the manner described with reference to FIGS. 1A-1B and FIG. 2. In this example, the flowcell used the reversible linkage between biotin and streptavidin, and used the biotin pre-grafted polymer approach described with reference to FIG. 1B, biotin was introduced on FC surface directly during the coating step.

Biotin pre-grafted polymers were synthesized modifying a precursor polyacrylamide polymer, which included azide groups, with a biotin-PEG-BCN linker in the manner shown below:

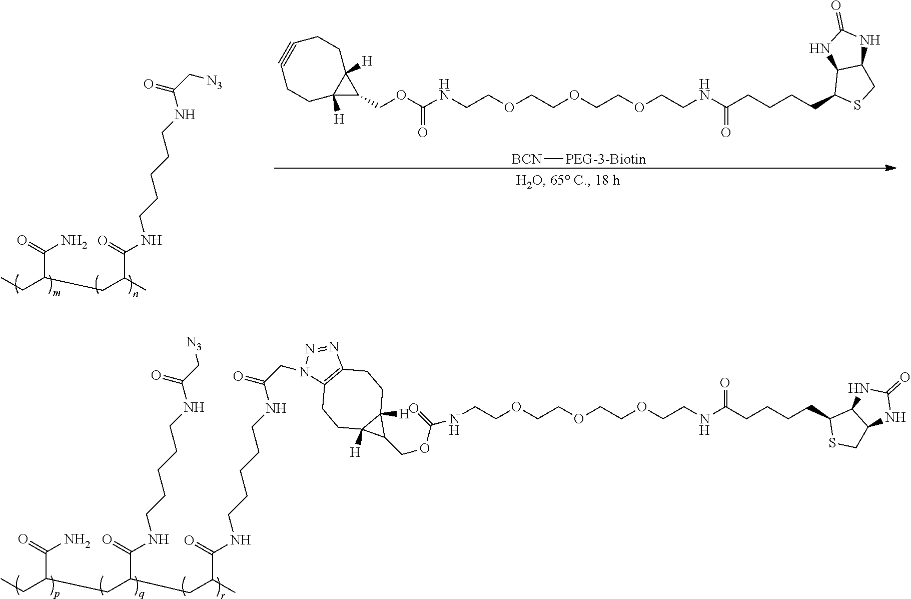

The amounts of biotin-linker were varied in order to obtain several example degrees of biotin functionalization of the polymer. More specifically, it was estimated that about 85% of the azide groups in the precursor polyacrylamide polymer were available for attachment (see WO 2013 184796 A1, the entire contents of which are incorporated by reference herein). Several individual polymers containing different percentages of grafted biotin were prepared in the manner described with reference to FIG. 1B. The polymers were purified by tangential flow filtration (TFF) and the percentage of biotin incorporation was determined by ¹H-NMR by comparing relevant signals of biotin with that of the polymer backbone. Table 3 shows the theoretical amount, and the experimentally measured amount, of biotin in each of these polymers. From this data, it may be understood that the experimentally measured amount of biotin was very close to that which was expected.

TABLE 3
Biotin incorporation as determined by 1H-NMR.

| | Theoretical | |
|---|---|---|
| | Biotin | Experimental Biotin |
| Polymer | (mol %) | ¹H-NMR (mol %) |
| A | 5 | —* |
| B | 15 | 13 |
| C | 25 | 23 |
| D | 35 | 32 |
| E | 50 | 49 |

*Small 1H-NMR integrals.

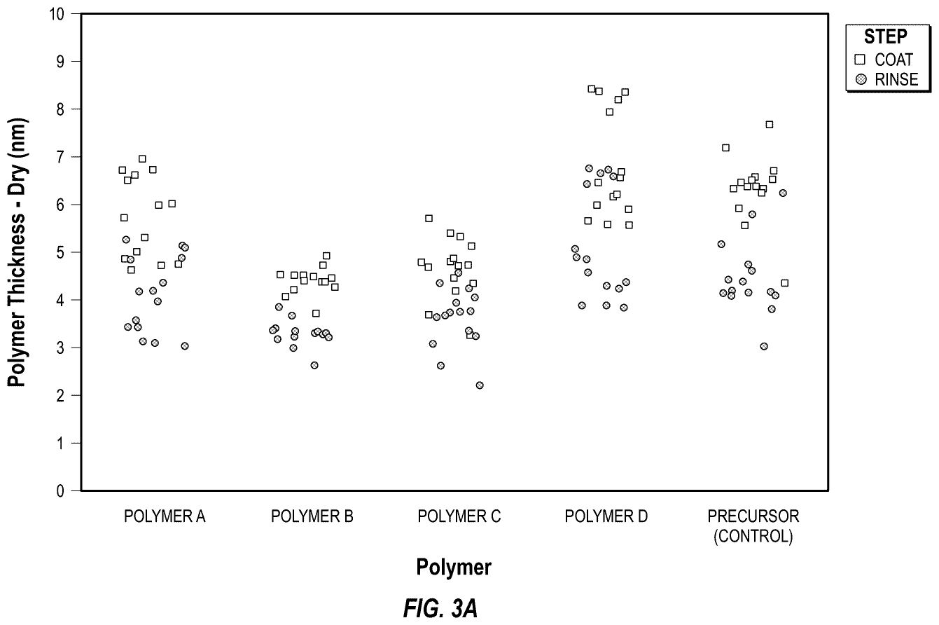

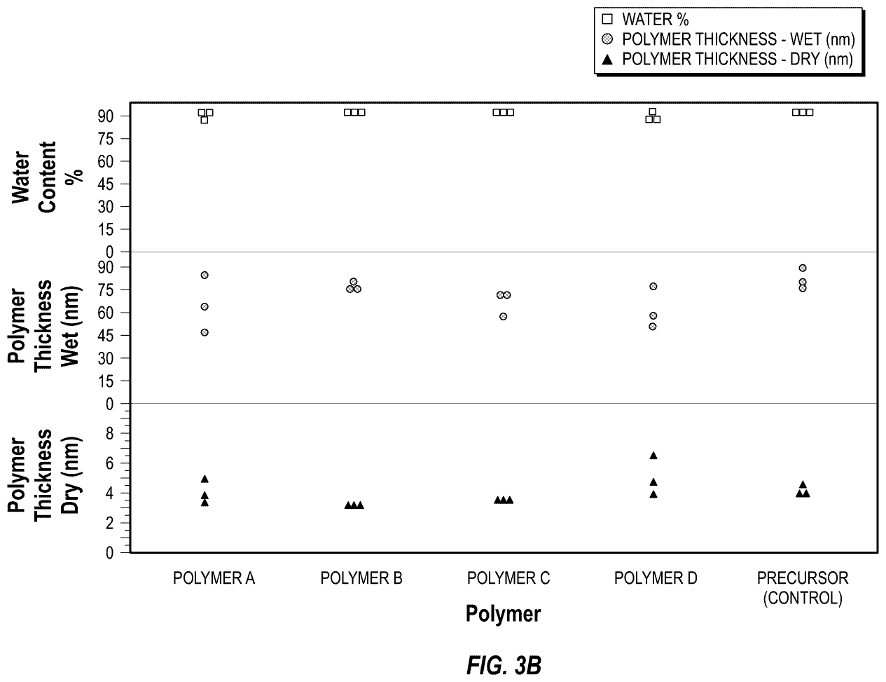

Coating performance was compared to that of precursor polymer. More specifically, the different polymer formulations, 0.2 wt % in water, were used to spin coat model TaOx slides which were intended to simulate the surface of a CMOS image sensor. Curing was performed at 80° C. for 10 minutes on a hotplate. All the tested polymers showed comparable thickness as determined by dry and wet ellipsometry (FIGS. 3A-3B), therefore confirming that polymer modification with biotin does not influence coating performance. More specifically, FIG. 3A is a plot of coating thickness before and after rinse in model unpatterned TaOx slides, and FIG. 3B is a plot of dry vs. wet thickness as measured by ellipsometry measurements. The biotinylated polymers A, B, C, and D of Table 3 were compared with the precursor polymer.

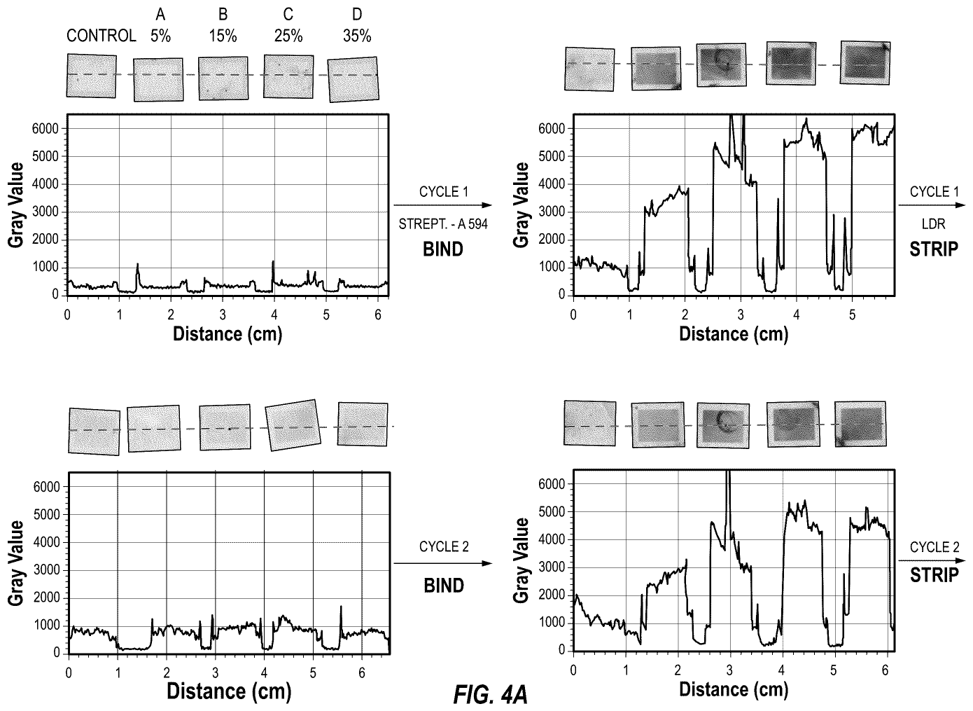

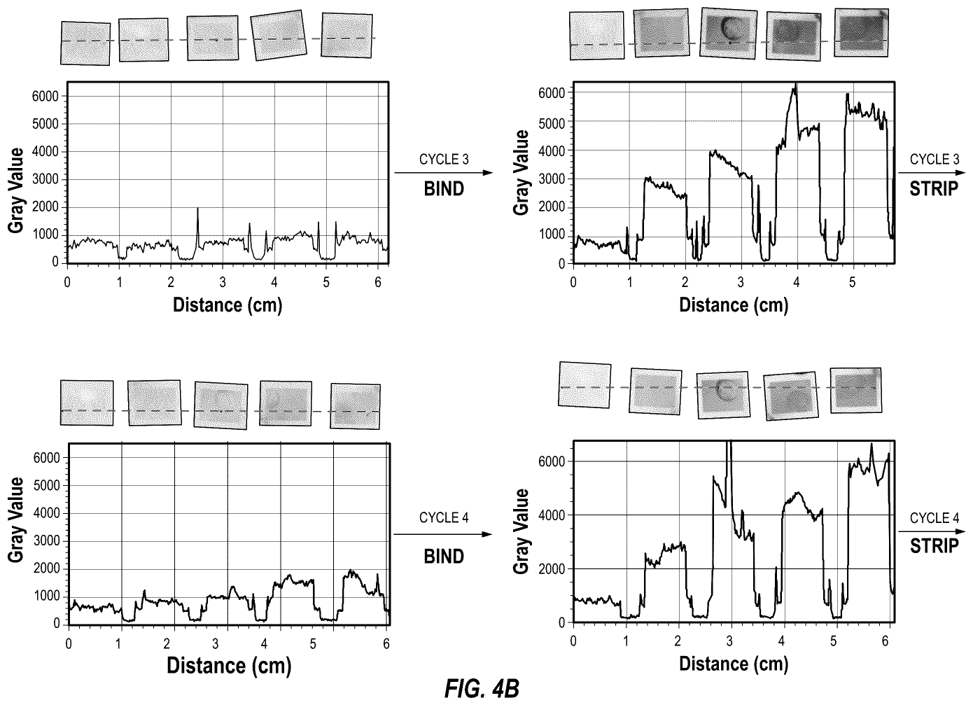

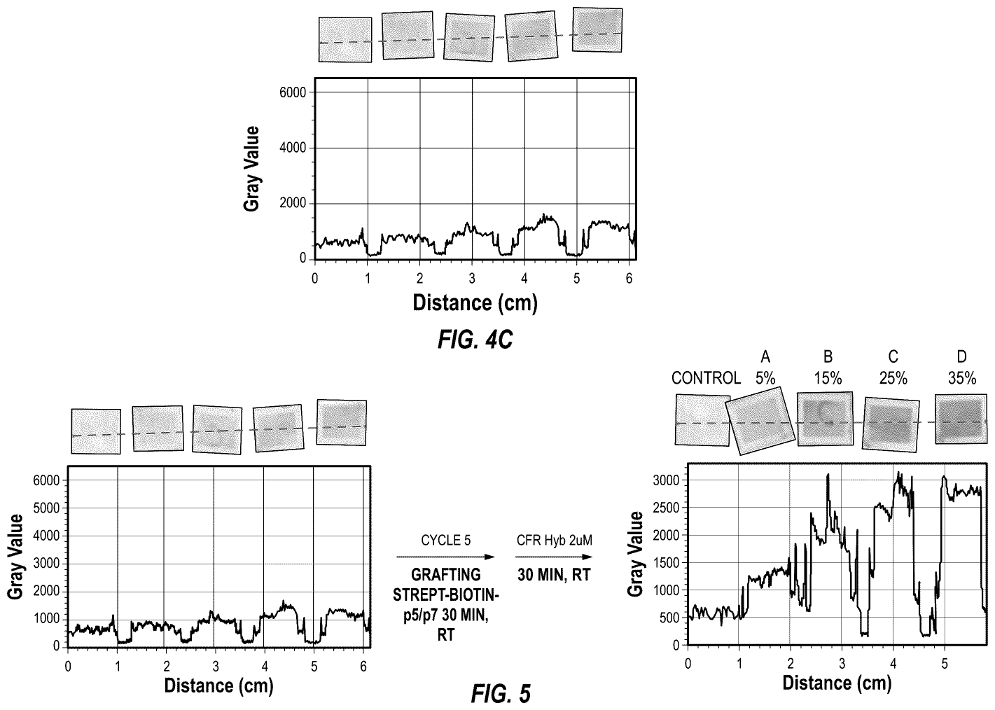

FIGS. 4A-4C illustrate fluorescence images and intensities obtained following binding steps and stripping steps of example polymers. More specifically, formulations A, B, C, D, and precursor polymer as a control, were used to coat single patterned CMOS dies and cured. The polymers then were incubated with a streptavidin-Alexa 594 (0.4 mg/mL) solution, at room temperature for 30 minutes (Bind Steps in FIGS. 4A-4B). Dies were rinsed in HT1 and imaged (TAMRA channel, fluorescence) on Typhoon. Higher biotin content led to higher streptavidin binding and therefore higher fluorescence intensity, as may be seen in both the images and intensities following the Bind Steps in FIGS. 4A-4B, confirming selective streptavidin binding to the surface. Streptavidin was then removed (Strip Steps in FIGS. 4A-B) in formamide at 70° C. for 15 minutes. As expected, fluorescence intensity dropped, indicating reaction reversibility (FIG. 4C).

FIG. 5 illustrates fluorescence images and intensities obtained after grafting oligonucleotides to example polymers. More specifically, the bind/strip steps of FIGS. 4A-4C were repeated 4 times and finally, at the fifth cycle, streptavidin dye was replaced by streptavidin-dual biotin p5/p7. CFR hybridization (2 uM) was then performed for 30 minutes at room temperature and the dies were washed and imaged (FIG. 5). CFR hyb refers to an assay where fluorescently labeled complements to P5 and P7 oligos are flushed into the flowcell and imaged in order to quantify the amounts of P5 and P7 oligos at the surface. From FIG. 5, it may be understood that the pre-grafted biotin polymer can reversibly bind oligonucleotides and that the number of oligonucleotides on the surface can be tuned by varying the amount of biotin on the polymer.

The number of primers per well and reaction reversibility were also assessed. More specifically, polymers A, B, and D and the precursor polymer were also used to coat a CMOS substrate. Streptavidin-dualbiotin P5/P7 was bound to the surface, then CFR Hyb was flushed through the flowcell, then an NaOH solution was used to dehybridize the oligonucleotides from the surface. Fluorescence of the collected NaOH solution was measured, and used to estimate the concentration of P5/P7 primers at the surface for each of the polymers. Higher biotin content was observed to result in a larger number of primers per well. Bind/strip operations such as described with reference to FIGS. 4A-4C were repeated several times to confirm reaction reversibility and flowcell regeneration. The strip cycles were observed to remove approximately 100% of the primers. These results confirm that the chemistry can be reversed several times (e.g., at least five times), and that the flowcell can therefore be reused.

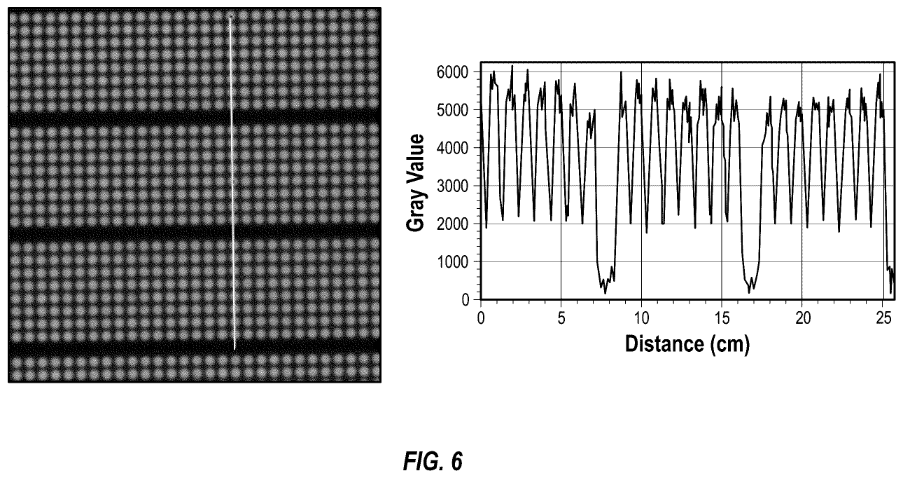

FIG. 6 illustrates fluorescence images and intensities obtained after disposing example polymer D in nanowells of a CMOS substrate. More specifically, FIG. 6 illustrates an image obtained using confocal fluorescent microscopy of polymer D after CFR Hyb, and the fluorescence intensity across the vertical line shown in the image. The polymer in the nanowells is light grey. This image shows that the polymer is confined in nanowells, and fluorescence intensity between nanowells is reproducible. The polymer was used for three sequencing runs, in between which the previously used oligonucleotides were stripped and a new set of oligonucleotides added. The sequencing performance was similar in all three runs, as shown in Table 4 below:

TABLE 4

| | PF | Occ | % Q30 |
|---|---|---|---|
| Run 1 | 43.58 | 86.66 | 85 |
| Run 2 | 44.91 | 87.52 | 89.5 |
| Run 3 | 42.71 | 88.03 | 79 |

From these results, it is shown that flowcells using the present polymers and methods may be satisfactorily reused for sequencing multiple times, e.g., at least three times.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of forming a sequencing flowcell, the method comprising:
in a solution, coupling a hydrogel precursor to a molecule by covalently bonding a first moiety of the hydrogel precursor to a second moiety of the molecule;
disposing the solution, including the hydrogel precursor coupled to the molecule, on a substrate to form a hydrogel disposed on the substrate; and
with the hydrogel disposed on the substrate, non-covalently bonding a third moiety of the molecule to a complex comprising an oligonucleotide.

2. The method of claim 1, wherein the substrate comprises an image sensor over which the hydrogel is disposed.

3. The method of claim 2, wherein the image sensor comprises a complementary metal oxide semiconductor (CMOS) sensor.

4. The method of claim 1, wherein the substrate comprises tantalum oxide (TaOₓ).

5. The method of claim 1, wherein the complex comprises a protein with a first active site to which the oligonucleotide is coupled, and a second active site that non-covalently bonds to the third moiety.

6. A sequencing flowcell, comprising:
an imaging sensor;
a hydrogel disposed on the imaging sensor and comprising a moiety;
a first complex non-covalently coupled to the moiety, the first complex comprising a first oligonucleotide.

7. A method of using the sequencing flowcell of claim 6, the method comprising:
decoupling the first complex from the moiety; and
coupling a second complex to the moiety, the second complex comprising a second oligonucleotide.
